(19) United States
(12) Patent Application Publication
(10) Pub. No.: US 2020/0318153 A1
(43) Pub. Date: Oct. 8, 2020

(54) Method and device of using aqueous two-phase systems (ATPS) for enhancing diagnostics for dental and oral diseases

(71) Applicant: PHASE DIAGNOSTICS, INC., Garden Grove, CA (US)

(72) Inventors: Yin To CHIU, Irvine, CA (US); Garrett Lee MOSLEY, Anaheim, CA (US); Brian Sangwoo LEE, La Mirada, CA (US)

(21) Appl. No.: 16/642,918

(22) PCT Filed: Aug. 30, 2018
(86) PCT No.: PCT/US2018/048802

§ 371 Date: Feb. 28, 2020

Related U.S. Application Data
(60) Provisional application No. 62/553,205, filed on Sep. 1, 2017.

(51) Int. Cl. C12Q 1/06; G01N 1/34

(52) U.S. Cl. CPC C12Q 1/06; G01N 1/34

(57) ABSTRACT

This invention relates to a method and device to improve the detection accuracy and performance for diagnosing dental disorders or diseases by improving the sensitivity of the Lateral-Flow Immunoassay (LFA). The present method and device are related to removing the protein interference (impurities) from sample and using aqueous two-phase system (ATPS) embedded entirely within a porous material, allowing spontaneous phase separation and concentration, for detection using the Lateral-Flow Immunoassay (LFA). The present invention also provides a platform technology for screening different types of specimens with increased sensitivity, and screening antibodies for optimal detection in various types of samples.

### CROSS-REFERENCE TO RELATED APPLICATIONS

This application claims the benefit of U.S. Provisional Application No. 62/553,205, filed Sep. 1, 2017. The entire contents and disclosures of the preceding application are incorporated by reference into this application.

Throughout this application, various publications are cited. The disclosures of these publications in their entireties are hereby incorporated by reference into this application to more fully describe the state of the art to which this invention pertains.

### STATEMENT REGARDING FEDERALLY SPONSORED RESEARCH OR DEVELOPMENT

This invention was made at least in part with government support under National Institutes of Health (NIH). The United States Government may have certain rights in the invention.

### FIELD OF THE INVENTION

This invention relates to a method and device to improve the detection accuracy and performance for diagnosing dental disorders or diseases by improving the sensitivity of the Lateral-Flow Immunoassay (LFA). The present method and device are related to removing the protein interference (impurities) from sample and using aqueous two-phase system (ATPS) embedded entirely within a porous material, allowing spontaneous phase separation and concentration, for detection using the Lateral-Flow Immunoassay (LFA). The present invention also provides a platform technology for screening different types of specimens with increased sensitivity, and screening antibodies for optimal detection in various types of samples.

### BACKGROUND OF THE INVENTION

Dental caries, also known as tooth decay or cavities, is the most common oral disease across the globe, affecting 2.4 billion people. Over 92% of the population have dental caries at some point in their lifetime, even in industrialized nations with good healthcare systems. Restorative treatment of dental caries has been used for decades; however, it is estimated that 71% of all restorations are performed on previously restored teeth, demonstrating that the root cause is not being addressed.

In recent years, a better understanding of the caries process has changed the current restorative treatment philosophy. Personalized preventive strategies are preferred to restorative treatments unless the carious lesion has reached cavitation. This can be accomplished through identifying and decreasing caries risk factors.

Extensive research over the past few decades has demonstrated that dental caries is a result of microbial infection by cariogenic bacteria, predominately Streptococcus mutans (SM). Studies have evaluated and validated SM concentration levels as a predictive factor for caries (Loesche, W. J., Microbiol. Rev.50:353-380, 1986). The predictive value of SM levels can therefore be utilized for preventative care through proper risk monitoring and management. For this reason, several methods have been used to quantify the concentration of these cariogenic bacteria. One of the methods is to plate out and cultivate the sample or salvia containing the cariogenic bacteria on nutrient media. However, this method is relatively time-consuming which requires the cultivation and counting the number colonies of cariogenic bacteria. The whole process including the cultivation procedure and result analysis take at least few days to complete.

Although the prior art has furthered the understanding of caries development and methods of quantitative measurement of concentration of cariogenic bacteria, there is still a need to further enhance the sensitivity of the caries determination and convenience of the detection.

Lateral flow assay (LFA) is one of the popular detection tool widely used in in detecting cariogenic bacteria since it is rapid and easy to use. However, LFA test are generally only capable of providing qualitative results, not quantitative result, due to the limit of detection. A method to concentrate the cariogenic bacteria by porous material embedded with ATPS has been disclosed in patent WO2017041030. Unfortunately, the fold of concentration was only about 10 to 60-fold for detecting Streptococcus mutans (SM) on, which is the dominant bacterium that could lead to dental caries (cavities). The low concentration hardly afford an accurate and sensitive diagnosis of dental caries. An improved method and device to achieve more than 60-fold concentration is highly desired.

To overcome these limitations, the present invention provides an improved method and device to improve the detection limit of LFA by purifying and concentrating the cariogenic bacteria for further analysis on lateral flow assay (LFA) by removing the protein interference (impurities) from sample and using aqueous two-phase system (AWS) embedded entirely within a porous material, allowing spontaneous phase separation and concentration. The present invention can be easily and quickly done in one or two step without the need of complex equipment. The present method and device can improve the detection limit of LFA up to 100× or more.

Overall, the methods and device described herein can improve the accuracy, sensitivity and efficiency of the detection and quantification of cariogenic bacteria and therefore are capable of improving the performance of various analytical or diagnostic technologies relying on the detection and/or quantification of cariogenic bacteria. Many related diseases are cured if the diseases are detected early.

### SUMMARY OF THE INVENTION

This invention relates to a method and device to improve the detection accuracy and performance for diagnosing dental disorders or diseases by improving the sensitivity of the Lateral-Flow Immunoassay (LFA).

This invention relates to a method and device to improve the detection accuracy and performance for diagnosing dental disorders or diseases by improving the sensitivity of the Lateral-Flow Immunoassay (LFA). The present method and device are related to removing the protein interference (impurities) from sample.

The method of the present invention is based on an aqueous two-phase system (ATPS) embedded entirely within a porous material, allowing spontaneous phase separation and concentration.

The present invention relates to provides a platform technology for screening different types of specimens with increased sensitivity, and screening antibodies for optimal detection in various types of samples. With integration of Streptococcus mutans (SM) lateral-flow immunoassay (LFA) test and optimized porous material module material, the performance has been further enhanced.

The detection limit of LFA developed by the present invention can be improved up to 100× or more.

### BRIEF DESCRIPTION OF THE DRAWINGS

FIGS. 1A-1B show the concentration induced by the addition of ATPS components (e.g. polymer and salt) to saliva supernatant. Tube in FIG. 1A consists of saliva supernatant and ATPS components mixture with a volume ratio of 1:1. By adjusting ATPS components, the volume ratio of top phase to bottom phase can be changed from 1:1 to 9:1, and can be further altered to concentrate a target molecule into a phase with a smaller volume. FIG. 1B shows that a typical target molecule (shown in darker colour) was concentrated in the bottom phase with a 9:1 volume ratio.

FIG. 2 shows phase separation with different dental specimens. Panel A of FIG. 2 shows phase separation in plaque specimens, and Panel B of FIG. 2 shows the whole saliva specimens from different donors. Top panel in each figure shows the mixed phase solution prior to phase separation. Bottom panel in each figure shows final state of phase separated solution with the concentrated phase (potentially containing analyte) indicated by a darker color (shown in the bottom phase) with the other phase indicated by a lighter color (shown in the top phase). All tested samples achieved at least a 9:1 volume ratio, which is indicated by the dashed line.

FIG. 3 shows a LFA using a promising antibody pair in both plaque specimen buffer (PSB) and spun saliva. Samples were spiked with the indicated concentrations of SM. The presence of a test line (test) denotes a positive result, and the presence of a control line (ctrl) indicates a valid test. The detection limit for this test was at 10⁶ CFU/mL.

FIG. 4 shows a LFA using a promising antibody pair in both plaque specimen buffer (PSB) and spun saliva after ATPS concentration. Samples were spiked with the indicated concentrations of SM. The presence of a test line (test) denotes a positive result, and the presence of a control line (ctrl) indicates a valid test. The detection limit for this test was at 104 CFU/mL.

FIG. 5 shows the spontaneous phase separation of a solution using a paper strip with dehydrated ATPS components. Initially as illustrated in the first column of the upper panel, the gold nanoparticles (GNs) were rehydrated by a solution containing a blue dye. The dye and GNs were well mixed as the solution rehydrated the ATPS components. The solution then phase separated, resulting in a clear purple leading front while the blue dye is held back.

FIG. 6 shows the test results from a one-step porous material/LFA device in both PSB and spun saliva samples spiked with the indicated concentrations of SM. The presence of a test line (test) indicates a positive result, and the presence of a control line (ctrl) indicates a valid test. The detection limit for this test was at 10⁴ CFU/mL.

FIG. 7 is a schematic diagram of LFA outcomes for a semi-quantitative test. The three potential outcomes of the diagnostic test (low, moderate and high risk) as shown will allow clinicians to make a risk assessment and treatment decision based on the SM burden in the context of other factors. As shown in Panel (a) of FIG. 7, the presence of only one line indicates that the SM concentration in sample solution is lower than 10⁴ cfu/ml (low risk of caries). As shown in Panel (b) of FIG. 7, the presence of two lines indicates that the SM concentration in sample solution ranges from 10⁴ cfu/ml to 10⁶ cfu/ml (moderate risk of caries). The presence of three lines as shown in Panel (c) of FIG. 7 indicates that the SM concentration in sample solution is higher than 10⁶ cfu/ml (high risk of caries).

FIG. 8 shows the results from a semi-quantitative test for SM infection in spun saliva. The presence of a control line (ctrl) indicates a valid test, and in this case, the SM burden is less than 10⁴ CFU/mL. The presence of the control and first test line indicated SM burden >10⁴ CFU/mL, but <10⁶ CFU/mL, while the presence of all three lines indicated SM burden >10⁶ CFU/mL. All three risk levels were detected and represented in this figure.

FIG. 9 demonstrates the mechanism of semi-quantitative lateral flow assay using sandwich format according to one embodiment of the present invention (a: sample application pad, b: conjugate pad, c: nitrocellulose membrane, d: test line, e: test line, f: control line, g: adsorbent pad). Panel A of FIG. 9 shows when sample having a SM concentration of lower than 10⁴ cfu/ml is applied onto the LFA strip, only one line will be visible as the GNs are concentrated at control line (f). Panel B of FIG. 9 shows when sample having a SM concentration of 10⁴ cfu/ml to 10⁶ cfu/ml is applied onto the LFA strip, two lines will be visible at test line (e) and control line (f). Panel C of FIG. 9 shows when sample having a SM concentration of higher than 10⁶ cfu/ml is applied onto the LFA strip, all three lines will be visible at test line (e), test line (d) and control line (f). In one embodiment, Ab1 is antibody (or antigen) that can specifically bind to the target molecules; Ab2 is the antibody (or antigen) that can bind specifically to Ab1; and NP is gold nanoparticle (GNs) conjugated with the antibodies that can bind specifically to the target molecules. The sizes of GNs and antibody in the figure are not proportional.

FIG. 10 shows various examples of tapered porous material with geometries for different 3D paper well which is integrated into a sample application pad.

### DETAILED DESCRIPTION OF THE INVENTION

In the following description, several embodiments of the invention are described. For purposes of explanation, specific configurations and details are set forth in order to provide a thorough understanding of the embodiments. In addition, to the plural or singular forms of a word and to the extent that orientations of the embodiments are described as, “top”, “bottom”, “front” “back”, “left”, “right” and the like, these wordings are to aid the reader in understanding the embodiments and are not meant to be limiting physically. It is apparent to a person skilled in the art that the present invention may be practiced without specific details. The invention will be better understood by reference to the examples which follow, but those skilled in the art will readily appreciate that the specific examples are for illustrative purposes only and should not limit the scope of the invention which is defined by the claims which follow thereafter. It is to be noted that the transitional term “comprising” or “including”, which is synonymous with “containing” or “characterized by”, is inclusive or open-ended and does not exclude additional, unrecited elements or method steps.

The present invention relates to a method and device to improve the detection accuracy and performance for diagnosing dental disorders or diseases by diseases by improving the sensitivity of the Lateral-Flow Immunoassay (LFA).

The present invention relates to a method and device to remove the protein interference (impurities) from sample. The method and device also use an aqueous two-phase system (ATPS) embedded entirely within a porous material, allowing spontaneous phase separation and concentration, for detection using the Lateral-Flow Immunoassay (LFA).

The present invention provides an ATPS platform technology that allows the simultaneous analysis of both saliva and plaque specimens. In one embodiment, the adaptation of the present ATPS to both saliva and plaque specimens is achieved. These newly developed ATPSs, which spontaneously concentrate Streptococcus mutans (SM), successfully push forward and develop the two-step test for the detection of SM, whereby a specimen was first mixed with ATPS components before analysis. In one embodiment, the present invention was able to achieve a 100-fold improvement in the detection of SM in both saliva and plaque samples relative to normal LFA. In another embodiment, the porous material module's geometry and material, and the LFA test's antibodies were optimized to further enhance the performance. The present invention established a one-step test wherein the ATPS is fully integrated into a porous material module and does not require any liquid handling steps. The one-step test was also able to achieve a 100-fold improvement in both saliva and plaque samples.

In one embodiment, the present invention can reproducibly generate ATPSs with extreme volume ratios in saliva and plaque specimens of different origins.

### Type of Samples and Target Analytes

This invention relates to a method and device to improve the detection accuracy and performance for diagnosing dental disorders or diseases

This invention relates to a method and device to improve the detection accuracy and performance for diagnosing dental disorders or diseases from saliva.

Saliva is clear, viscous fluid with a slightly alkaline pH. It is hypotonic, composed of about 99.5% water, and also contains ions (e.g., K⁺, Na⁺, Ca²⁺, Mg²⁺, H+, Cl⁻, HCO₃⁻, I⁻, F⁻, HPO₄²⁻), and small organic molecules (e.g., ureas, hormones, lipids, DNA, and RNA). There are multiple contributors to the composition of saliva. Saliva has a complex “proteome”-106 D glycoproteins to 1000 D peptides. It contains secretory products of salivary glands, products of B cells, PMNs, epithelial cells, and bacteria. Major (e.g., parotid, submandibular, and sublingual) and minor (e.g., palatine and retromolar) glands contribute to the composition of saliva, along with extraneous contributors such as gingival crevicular fluid, serum proteins, white blood cells and their byproducts, oral epithelial cells, oral bacteria, food debris and dissolved food components. In one embodiment, the present methods can separate a target biomarker from non-target molecules (e.g., small molecules and macromolecules which are typically of natural origin and may interfere with the detection or quantification of target biomarker) in a saliva sample and thereby allows a more accurate detection and diagnosis.

In one embodiment, the present invention can be adapted to detect pathogens and bacteria that cause oral/dental disorders or diseases.

In one embodiment, the pathogens and bacteria that can be detected by the present invention include, but are not limited to, cariogenic bacteria, such as Streptococcus mutans (SM), Genus lactobacillus, Aggregatibacter actinomycetemcomitans, Porphyromonas gingivalis, Tannerella forsythia (formerly Bacteroides forsythus), Treponema denticola, Fusobacterium nucleatum, Prevotella intermedia, Prevotella nigrescens and Eikenella corrodens.

Purifying and Removing the Protein Interference (Impurities) from Saliva

This invention relates to a method and device to improve the detection accuracy and performance for diagnosing dental disorders or diseases by improving the sensitivity of the Lateral-Flow Immunoassay (LFA). The present method and device are related to removing the protein interference (impurities) from sample.

It is surprising that protein in the saliva will affect the sensitivity of LFA detection. In LFA detection, specific and effective binding is curial between antibodies and target antigen. However, the complex protein in saliva may interfere the LFA by falsely binding to the antibodies as antigen. As a result, the amount of actual antigen captured by antibodies was significantly reduced and the detection result will be far more lower than expected.

In the present invention, the complex protein in saliva sample was removed effectively, as a result, the specific and effective binding between antibodies and target antigen is enhanced in LFA detection. As a result, the detection limit was enhanced up to 100-fold in this invention.

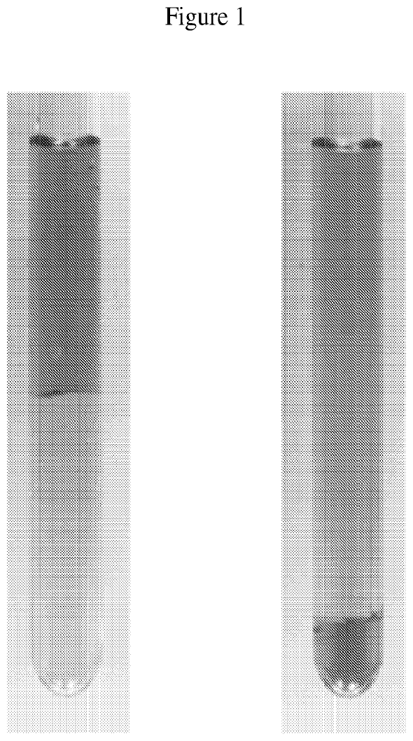

There are various methods available to remove protein from saliva by purifying agents which include, but are not limited to, trichloroacetic acid (TCA), 20% (w/v) TCA in acetone with 20 mM dithiothreitol (DTI), 20% (w/v) TCA in acetone with 0.07% (v/v) 2-mercaptoethanol, acetone and alcohol. Most of them involved the use of organic solvent which may affect the activity of target cariogenic bacteria. After screening, Trichloroacetic acid (TCA) was found to be the most suitable method. Conventional organic solvents such as acetone or alcohols can affect the activity of target cariogenic bacteria, and may even precipitate or damage the target bacteria or antigen. In one embodiment, the use of TCA as purifying agent does not precipitate or damage the bacteria or bacterial antgens. In one embodiment, the use of TCA as purifying agent well maintains the activity of the target analyte while interfering molecules are substantially removed from the solution. The following procedure can be followed: 500 μL of whole saliva specimens was mixed with 500 μL of TCA (20% w/v) and the mixture was vortexed to mix thoroughly and allowed to precipitate for 2 hour at −20° C. This was followed by centrifugation at 15,000 rpm, 4° C. for 20 min. The supernatant was collected. Sodium tetraborate was added to neutralization until slightly alkaline such as a pH value of 7.5. The supernatant can then be concentrated by lyophilizer or ATPS system as demonstrated in FIGS. 1A-1B if needed.

The assembly of the test strip comprises: a sample application pad (or a 3D paper well), a conjugate pad, a nitrocellulose membrane and an adsorbent pad. One or more test and control lines are covered on the nitrocellulose membrane.

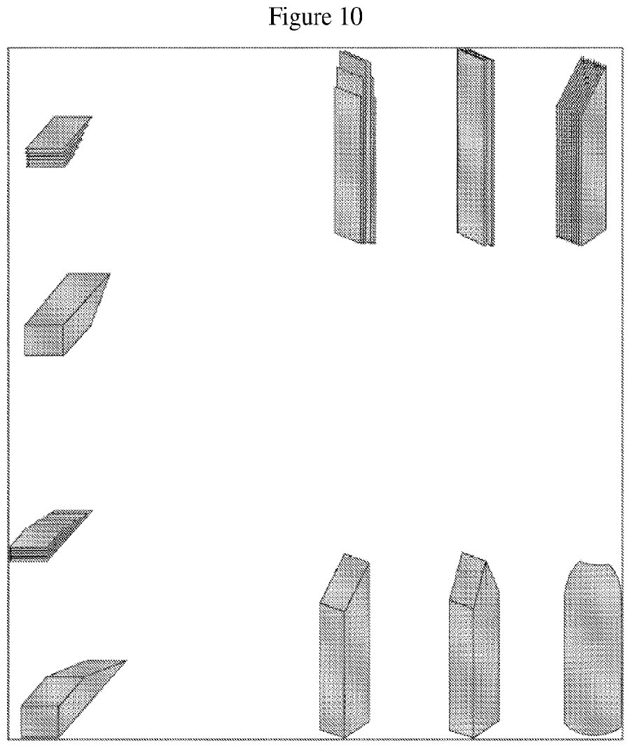

In this invention, the sample application pad (3D paper well) is made of porous material (fiber glass paper) embedded with ATPS for concentration purpose. Paper pieces can be stack together (3D paper well, as shown in FIG. 10) to increase the cross-sectional area so as to further enhance the concentration. Sample or purified sample is applied on this pad to start assay. The fluid is to migrate the fluid containing the analyte to other components of lateral flow test strip (LFTS). Sample pad should be capable of migrating fluid in a smooth, continuous and homogenous manner. In this invention, sample application pad was designed to concentrate the target analyte before its transportation by embedded ATPS on the porous material. In one embodiment, the sample pad is not separated from the 3D paper well. In one embodiment, a 3D paper well is part of the sample pad. In one embodiment, a 3D paper well is integrated the onto the sample pad.

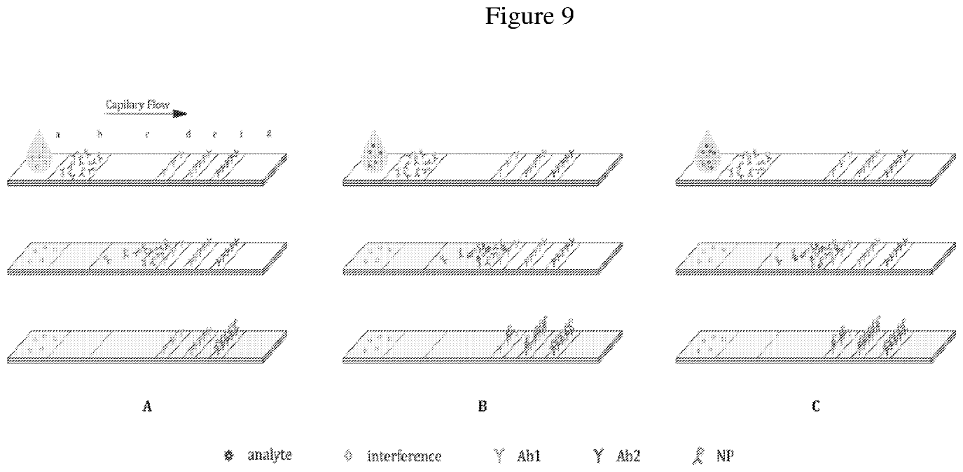

In one embodiment, the removal of interfering proteins is conducted on the sample application pad. In one embodiment, the precipitate formed between interfering proteins and purifying agent is retained at the sample pad and will not migrate with the flow of the liquid as shown in FIG. 9.

In one embodiment, the assembly may or may not contain a separate conjutate pad. In one embodiment, the conjugate pad is integrated as part of the sample application pad. In one embodiment, conjugate pad is the place where labeled biorecognition molecules are dispensed. Material of conjugate pad should immediately release labeled conjugate upon contact with moving liquid sample. Labeled conjugate should stay stable over entire life span of lateral flow strip. Any variations in dispensing, drying or release of conjugate can change results of assay significantly. Poor preparation of labeled conjugate can adversely affect sensitivity of assay. Glass fiber, cellulose, polyesters and some other materials are used to make conjugate pad for LFA. Nature of conjugate pad material has an effect on release of labeled conjugate and sensitivity of assay. Colloidal gold nanoparticles was labeled as indicator-containing (colloidal gold).

Nitrocellulose membrane is highly critical in determining sensitivity of LFA. Nitrocellulose membranes are available in different grades. Test and control lines are drawn over this piece of membrane. So an ideal membrane should provide support and good binding to capture probes (antibodies etc.). Nonspecific adsorption over test and control lines may affect results of assay significantly, thus a good membrane will be characterized by lesser non-specific adsorption in the regions of test and control lines. Wicking rate of nitrocellulose membrane can influence assay sensitivity. These membranes are easy to use, inexpensive, and offer high affinity for proteins and other biomolecules. Proper dispensing of bioreagents, drying and blocking play a role in improving sensitivity of assay. (Sajid M., et al., Journal of Saudi Chemical Society (2015) 19, 689-705)

Adsorbent pad works as sink at the end of the strip. It also helps in maintaining flow rate of the liquid over the membrane and stops back flow of the sample. Adsorbent capacity to hold liquid can play an important role in results of assay.

In one embodiment, the test strip is configured as a sandwich format. In one embodiment, the test strip is configured as a competitive format. In one embodiment, the test strip is configured as a multiplex detection format that can detect multiple analytes during one test.

### ATPS (Aqueous Two-Phase System)

This invention relates to a method and device to improve the detection accuracy and performance for diagnosing dental disorders or diseases by improving the sensitivity of the Lateral-Flow Immunoassay (LFA). The present method and device are related to removing the protein interference (impurities) from sample and using aqueous two-phase system (ATPS) embedded entirely within a porous material, allowing spontaneous phase separation and concentration, for detection. In one embodiment, the removal of interfering molecules is conducted on a purifying unit. In one embodiment, the concentration of target analyte occurs on the sample application pad. In one embodiment, the removal of interfering molecules and the concentration of target analyte are conducted on a sample application pad. In one embodiment, “removing interfering proteins (molecules)” refers to a purified status of a solution that does not physically contain the interfering proteins (molecules). In one embodiment, “removing interfering proteins (molecules)” refers to an outcome that the interference affect due to the interfering proteins (molecules) is prevented or minimized regardless whether the interfering proteins (molecules) or the products between the interfering proteins and the purifying agent are physically removed or not. In one embodiment, a precipitate is formed between the interfering proteins and the purifying agent. In one embodiment, the precipitate does not migrate with liquid flow. In one embodiment, the precipitate is immobilized on the sample application pad, the concentrating module, conjugate pad, and/or another separate pan that can immobilization or separate the precipitate from the liquid to be tested. In one embodiment, the precipitate does not affect the detection of the target analyte.

In one embodiment, there is provided a two-component ATPS for purifying a target analyte from a sample and concentration of the analyte. Different molecules in a mixture would be distributed differentially between the two phase solutions due to their different properties, and it is possible to separate and concentrate target molecules using ATPS with minimal set up and human intervention. No power or equipment is necessary to bring about the phase separation, as the fluid flow relies purely on capillary action which is based on isothermal-dynamic principles.

The advantage of the invention is that high purity and concentration of the target analyte can be obtained in a simple way and compatible with downstream application using Lateral-Flow Immunoassay (LFA) without further step of purification or concentration.

The methods and devices provided herein are robust, inexpensive, simple, easy to handle, safe, user friendly and fast. The present method is able to purify and concentrate the target analyte and thereby ensures the performance of the downstream applications using the purified and concentrated analyte will not be affected by impurities in the original sample.

Because of the unique features described herein, the present invention can purify and concentrate the target analyte conveniently and rapidly without the use of external power source or complex instrumentation, and is applicable to samples containing the target analyte in a very low amount, or of a small volume. Furthermore, the present method is readily adaptable to automation including high throughput screening systems.

In one embodiment, the present method is used to purify and concentrate a target analyte from saliva. The present method is able to separate the target analyte from non-target molecules. and concentrate the target analyte simultaneously.

In another embodiment, the present method is used to purify and concentrate a target analyte from plaque.

In one embodiment of the present method, the target analyte is retained on the ATPS while non-target materials are left in the liquid system (i.e., original sample plus any non-ATPS components).

### Design of ATPS-Embedded Porous Material

In one embodiment, the present invention provides a porous material embedded with ATPS components. Various ATPS systems can be used in the present invention, including but are not limited to polymer-polymer (e.g. PEG-dextran), polymer-salt (e.g. PEG-salt), and micellar (e.g. Triton X-114). Porous material may be made of any suitable porous material which can absorb and transfer liquid. Suitable porous materials for this invention include but are not limited to fiberglass paper, cotton-based paper, other types of paper, polymer foams, cellulose foams, other types of foams, rayon fabric, cotton fabric, other types of fabric, wood, stones, and any other materials that can absorb and transfer liquid.

In one embodiment, the ATPS comprises a mixed phase solution comprising a first phase solution and a second phase solution, wherein components of said first phase solution and components of said second phase solution are embedded in said porous material at a concentration or a loading that is sufficient to undergo a phase separation as the mixed phase solution flows through the porous material.

In one embodiment, components of the first phase solution and/or the components of said second phase solution of the ATPS are embedded in the porous material and then dehydrated prior to the addition of a sample containing the target analyte to said porous material.

In one embodiment, components of the first phase solutions and/or the components of said second phase solution of the ATPS are combined with a sample containing the target analyte to create a mixture prior to the addition of said mixture to the porous material.

In one embodiment, some of the components of the first phase solution and/or the components of the second phase solution of the ATPS are embedded in the porous material and then dehydrated, while the remaining components of the first phase solutions and/or the components of the second phase solution are combined with a sample containing the target analyte to create a mixture prior to the addition of the mixture to the porous material.

In one embodiment, there is provided a two-component ATPS (aqueous two phase system) within a porous material for the concentration of one or more target analytes and/or the purification of a sample solution. The target analyte is in contact with the mixed phase solution comprising a first phase solution and a second phase solution, and partitions into the first phase solution, the second phase solution or the interface (or interphase) between the first phase solution and the second phase solution.

In one embodiment, there is provided a two-component ATPS (aqueous two-phase system) within a porous material for removing one or more contaminants from a sample, thereby obtains a purified sample of the target analyte(s). In one embodiment, the one or more contaminants are in contact with the mixed phase solution comprising a first phase solution and a second phase solution, and wherein the contaminants partitions into the first phase solution, the second phase solution, or the interface (or interphase) between the first phase solution and the second phase solution.

In one embodiment, the porous material and ATPS are selected so that the first phase solution flows through the porous matrix at a first rate and the second phase solution flows through the porous matrix at a second rate, wherein the first rate and the second rate are different.

In one embodiment, the porous material is commercially available or manufactured in-house.

### Adjustment of Concentration Factors

In one embodiment, the relative amounts of ATPS components can be changed. The volume ratio of the two components of ATPS are controlled so as to concentrate the target analyte preferentially in one component.

To better quantify the phenomena associated with the present invention, an assay was developed to evaluate the correlation between the relative amounts of ATPS components and the fold of concentration achieved. With this, the concentration factor can be selected and fine-tuned by adjusting the relative amount of the ATPS components as needed.

In one embodiment, the ratio between the two phases in an ATPS can be easily controlled by varying concentrations of ATPS components. FIGS. 1A-B show phase separation concentration induced by the addition of different ATPS components (e.g. polymer and salt) to saliva supernatant. ATPS components and saliva supernatant were mixed 1:1. By adding ATPS components, the mixture phase separates and the target molecules partition into one of the two phases. Panel B shows that the target molecule was concentrated in the bottom phase with a 9:1 volume ratio. The volume ratio of top phase to bottom phase can be further altered to concentrate the target molecule in a phase with a smaller volume. In another embodiment, by adjusting the ATPS components (for example, adding additional ATPS component(s) to the mixture, or altering the relative volume or concentration of ATPS components in the mixture), the volume ratio of top phase to bottom phase can be changed from 1:1 to 9:1, or higher ratios. This phenomenon can be leveraged to concentrate a target molecule without the need for power, equipment, or training. In a simple medium, such as water or a saline solution, this is trivial to reproduce; however, given a complex medium (e.g. saliva) that has high variability and potentially other interfering substances, it is more difficult to achieve a useful volume ratio with samples of different origins.

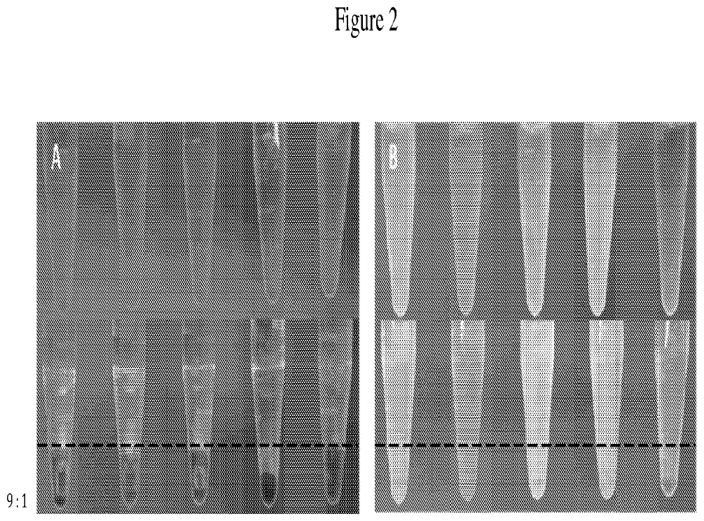

In one embodiment, through extensive screening, other than whole saliva specimens, the present invention can be applied for plaque specimen as well. FIG. 2 shows that concentration in different dental specimens including phaque and whole saliva specimens. Panel A of FIG. 2 shows the phase separation and concentration in plaque specimens, and Panel B of FIG. 2 shows that in whole saliva specimens from different donors. Top panel shows the mixed phase solution prior to phase separation and concentration. Bottom panel shows the final state of phase separated solution with the concentrated phase (potentially containing analyte) indicated by a darker color with the top phase indicated by a lighter color. All tested samples achieved a top/bottom volume ratio of at least a 9:1, which is indicated by the dashed line. These results demonstrated that the versatility and applicability of the ATPS system are for not only different patients, but also for different sample types as well.

In one embodiment, to integrate the ATPS components into the porous material, the ATPS components are solubilized in water (or appropriate buffer) and applied on the porous material in certain ratios and/or concentrations. The porous materials are then placed in a lyophilizer to remove water, resulting in the embedment of ATPS components directly on the porous material. Upon introduction of the sample to the porous materials, the ATPS components instantly undergo rehydration and thereby separate the molecules in the sample and concentrate the target analyte such as biomarker at the front of the fluid flow without any external power or equipment to provide a driving force.

In one embodiment, a porous fiberglass paper is impregnated with ATPS which is made of polymer to polymer based PEG-dextran. When a sample containing a plurality of biomarkers is poured onto the ATPS coated porous fiberglass paper, the impregnated porous fiberglass paper preferentially causes the biomarker and some ATPS components flow ahead of the other ATPS components. Therefore, the targeted biomarker is concentrated at the front of the fluid flow.

In one embodiment, the porous fiberglass paper is pretreated with ATPS components in a form of polymer, salt and polymer/salt solution. In one embodiment, the polymer is a PEG.

In one embodiment, a porous fiberglass paper is impregnated with ATPS components in a form of micell solution/emulsion/suspension containing surfactant.

In one embodiment, there are various ATPS systems including but are not limited to polymer-polymer (e.g. PEG-dextran), polymer-salt (e.g. PEG-salt), and micellar (e.g. Triton X-114). The first and/or second component comprises a polymer. Polymer includes but is not limited to polyalkylene glycols, such as hydrophobically modified polyalkylene glycols, poly(oxyalkylene)polymers, poly(oxyalkylene)copolymers, such as hydrophobically modified poly(oxyalkylene)copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, and poly N-isopropylacrylamide and copolymers thereof. In another embodiment, the first polymer comprises polyethylene glycol, polypropylene glycol, or dextran.

In one embodiment, the polymer concentration of the first component or second component is in the range of about 0.01% to about 90% by weight of the total weight of the aqueous solution (w/w). In various embodiments, the polymer solution is selected from a polymer solution that is about 0.01% w/w, about 0.05% w/w, about 0.1% w/w, about 0.15% w/w, about 0.2% w/w, about 0.25% w/w, about 0.3% w/w, about 0.35% w/w, about 0.4% w/w, about 0.45% w/w, about 0.5% w/w, about 0.55% w/w, about 0.6% w/w, about 0.65% w/w, about 0.7% w/w, about 0.75% w/w, about 0.8% w/w, about 0.85% w/w, about 0.9%) w/w, about 0.95% w/w, or about 1% w/w. In some embodiments, the polymer solution is selected from polymer solution that is about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, about 25% w/w, about 26% w/w, about 27% w/w, about 28% w/w, about 29% w/w, about 30% w/w, about 31% w/w, about 32% w/w, about 33% w/w, about 34% w/w, about 35% w/w, about 36% w/w, about 37% w/w, about 38% w/w, about 39% w/w, about 40% w/w, about 41% w/w, about 42% w/w, about 43% w/w, about 44% w/w, about 45% w/w, about 46% w/w, about 47% w/w, about 48% w/w, about 49% w/w, and about 50% w/w.

In one embodiment, the first and/or second component comprises a salt, the salt includes but is not limited to kosmotropic salts, chaotropic salts, inorganic salts containing cations such as straight or branched trimethyl ammonium, triethyl ammonium, tripropyl ammonium, tributyl ammonium, tetramethyl ammonium, tetraethyl ammonium, tetrapropyl ammonium and tetrabutyl ammonium, and anions such as phosphates, sulphate, nitrate, chloride and hydrogen carbonate. In another embodiment, the salt is selected from the group consisting of NaCl, Na₃PO₄, K₃PO₄, Na₂SO₄, potassium citrate, (NH₄)₂S₄, sodium citrate, sodium acetate and combinations thereof. Other salts, e.g. ammonium acetate, may also be used.

In one embodiment, the total salt concentration is in the range of 0.001 mM to 100 mM. A skilled person in the art will understand that the amount of salt needed to form an aqueous two-phase system will be influenced by molecular weight, concentration and physical status of the polymer.

In one embodiment, the first component and/or the second component in the ATPS comprises a solvent that is immiscible with water. In some embodiments, the solvent comprises a non-polar organic solvent. In some embodiments, the solvent comprises an oil. In some embodiments, the solvent is selected from pentane, cyclopentane, benzene, 1,4-dioxane, diethyl ether, dichloromethane, chloroform, toluene and hexane.

In one embodiment, the first component and/or second component in the ATPS comprises a micellar solution. In some embodiments, the micellar solution comprises a nonionic surfactant. In some embodiments, the micellar solution comprises a detergent. In some embodiments, the micellar solution comprises Triton-X. In some embodiments, the micellar solution comprises a polymer similar to Triton-X, such as Igepal CA-630 and Nonidet P-40. In some embodiments, the micellar solution consists essentially of Triton-X.

In one embodiment, the first component in the ATPS comprises a micellar solution and the second component in the liquid phase comprises a polymer. In one embodiment, the second component in the liquid phase comprises a micellar solution and the first component in the liquid phase comprises a polymer. In one embodiment, the first component in the liquid phase comprises a micellar solution and the second component in the liquid phase comprises a salt. In one embodiment, the second component in the liquid phase comprises a micellar solution and the first component comprises a salt. In one embodiment, the micellar solution is a Triton-X solution. In one embodiments, the first component comprises a first polymer and the second component comprises a second polymer. In one embodiment, the first/second polymer is selected from polyethylene glycol and dextran. In one embodiments, the first component comprises a polymer and the second component comprises a salt. In one embodiment, the second component comprises a polymer and the first component comprises a salt. In some embodiments, the first component comprises polyethylene glycol and the second component comprises potassium phosphate. In some embodiments, the second component comprises polyethylene glycol and the first component comprises potassium phosphate. In one embodiment, the first component comprises a salt and the second component comprises a salt. In one embodiments, the first component comprises a kosmotropic salt and the second component comprises a chaotropic salt. In some embodiments, the second component comprises a kosmotropic salt and the first component comprises a chaotropic salt.

### Improvement of Diagnostic Procedures Using the Lateral-Flow Immunoassay (LFA)

Purified saliva obtained by the present method can be subject to detection or analysis using the Lateral-Flow Immunoassay (LFA) for diagnosing dental disorders or diseases.

Lateral flow immunoassay (LFA) methods and devices have been described extensively. See, e.g., Gordon and Pugh, U.S. Pat. No. 4,956,302; H. Buck, et al., WO 90/06511; T. Wang, U.S. Pat. No. 6,764,825; W. Brown, et al., U.S. Pat. No. 5,008,080; Kuo and Meritt, U.S. Pat. No. 6,183,972, EP 00987551A3. Such assays involve the detection and determination of an analyte substance that is a member of a specific binding pair consisting of a ligand and a receptor. The ligand and the receptor are related in that the receptor specifically binds to the ligand, being capable of distinguishing a specific ligand or ligands from other sample constituents having similar characteristics. Immunological assays involving reactions between antibodies and antigens are one such example of a specific binding assay. Other examples include DNA and RNA hybridization reactions and binding reactions involving hormones and other biological receptors.

In one embodiment, analyte and/or analytes-containing solution obtained by the present invention can be analyzed by a lateral flow assay (LFA). LFA has a number of desirable characteristics including their ease of use and board applicability to a variety of analytes. However, LFA is generally only capable of providing qualitative results due to its detection limitation. For example, the detection limit of LFA on pathogen (S. mutans) is 10⁶ CFU/ml. In the present invention wherein protein in saliva sample was first removed as impurities, the detection limit of LFA on S. mutans can be improved to as low as 104 CFU/ml amounting to a 100-fold enhancement. Together with the improved concentration fold by 100-fold, the present invention is suited for providing quantitative result in a broader range.

In one embodiment, by adjusting the concentration of labeled biorecognition molecules (e.g., GNs) immobilized at the different lines, the concentration of target analyte is thus semi-quantitatively observed. The higher concentration of the labeled biorecognition molecules (GNs), the higher is the color intensity. In one embodiment, to adjust the concentration of GNs at the different lines, different concentration of corresponding antibodies (or antigens) will be prefixed onto one or more control or test lines on the nitrocellulose membrane. In one embodiment, the color of one test line is intense enough to be visible if the concentration of SM is higher than 10⁴ cfu/ml. In one embodiment, the color of two test lines is be intense enough to be visible if the concentration of SM is higher than 10⁶ cfu/ml. In one embodiment, the number of lines and the analyte concentrations are configured based on the correlation between a set of experiments. FIG. 9 demonstrates the mechanism of semi-quantitative lateral flow assay using one embodiment of the present invention.

In one embodiment, the present invention is capable of screening antibodies for optimal compatibility with saliva or plaque specimens. In one embodiment, the antibodies are commercially available antibodies or their derivatives. In another embodiment, the antibodies are newly developed and produced using methods conventionally adapted for the development and production of antibodies at the time of invention.

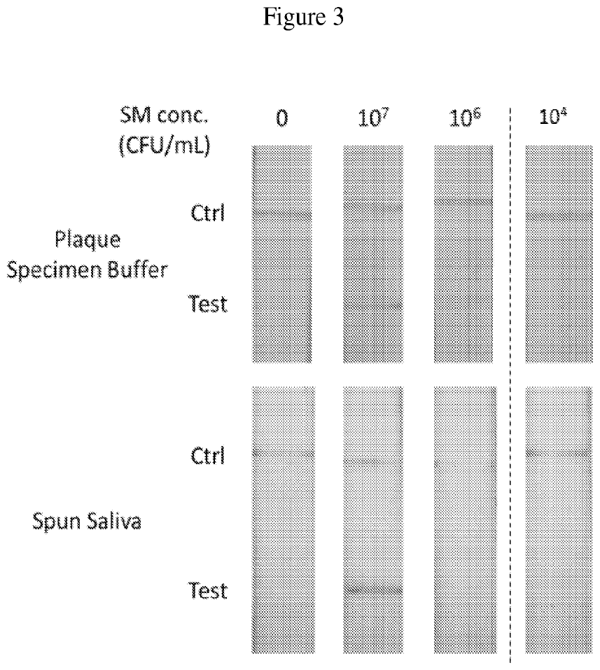

In one embodiment, the present invention screened antibodies in pairs in various types of samples and testing media. In one embodiment, the testing media is plaque specimen buffer (PSB) which is an aqueous buffer for dissolving plaque and saliva, and was used after the potential SM contamination was removed by centrifuge. In one embodiment, the antibodies pairwise (both conjugated to colorimetric indicator and immobilized on test strip) were tested in both PSB and spun saliva. Promising pairs were identified, as shown in Table 1. Many pairs did not bind to SM strongly enough (false negative) or displayed nonspecific binding (false positive). Three promising pairs including IgG, IgA and IgM classes of antibodies to Streptococcus mutans (two of which work in both sample types) were identified. A baseline detection limit of 10⁶ CFU/mL was established as shown in FIG. 3.

TABLE 1
Summary of antibody screening efforts in plaque specimens and saliva
specimens to identify promising pairs for further evaluation.

| Plaque Specimen Buffer | | | | Saliva Specimens | | | |
|---|---|---|---|---|---|---|---|
| | GN | | | | GN | | |
| M | Ab-A | Ab-B | Ab-C | M | Ab-A | Ab-B | Ab-C |
| Ab-A | *** | ** | * | Ab-A | *** | *** | ** |
| Ab-B | * | *** | * | Ab-B | * | *** | ** |
| Ab-C | ** | ** | ** | Ab-C | * | * | * |

* no or very faint test lines observed;
** nonspecific binding;
*** promising antibody pair for evaluation.
Ab: antibody; M: membrane; GN: gold nanoparticle. A: IgG; B: IgA; C: IgM.

In one embodiment, integration of ATPS with SM LFA test greatly improves limit of detection in a two-step process.

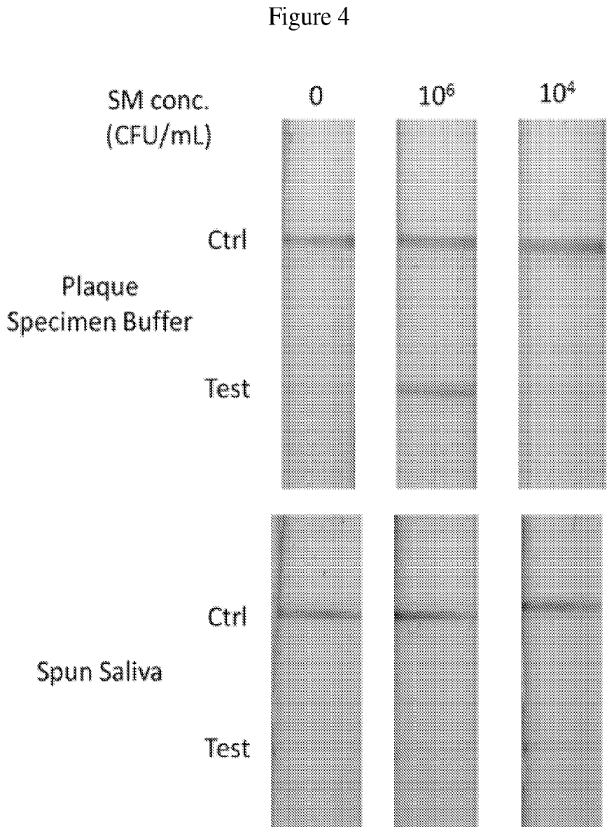

In one embodiment, with the critical components of the established device, ATPS and LFA are combined to achieve a higher sensitivity of detection. A two-step test format is implemented, whereby spun saliva or PSB is added to ATPS components and allowed to separate. The resulting phase containing the analyte of interest is then extracted and run on the LFA test strip. In one embodiment, the present invention is able to achieve a 100-fold improvement in the limit of detection of 10⁴ CFU/mL in both sample types (FIG. 4). Since the clinically relevant concentrations for SM are within the range of 10⁴ CFU/mL, the present invention is well-suited for clinical purposes.

In one embodiment, the porous material module material and geometry are modified to further optimize the performance of the present invention.

A two-step process consisting of the first step of using ATPS system to concentrate the target analyte and the second step of applying the concentrated analyte to LFA has been unutilized. In one embodiment, in order to improve on the success of the two-step system, a porous material module is utilized. While an ATPS typically requires steps of liquid handling, the porous material module would obviate the need for such steps and fully contain the actual phase separation/concentration phenomenon, thereby reducing user involvement and minimizing/avoiding the potential for user error and/or contamination. In one embodiment, an ideal porous material module would allow for a clean phase separation, good flow velocity, and proper orientation of phases (i.e. concentrated analyte is at the fluid front). In one embodiment, both the porous material and geometry of the porous material in the context of saliva and plaque specimens were optimized to enhance the performance. In another embodiment, it was found that the geometry made a large difference in fluid flow, as increasing the cross-sectional area while keeping the total amount of paper the same helped fluid flow, especially in whole saliva (Table 2). The cross-sectional area can be achieved by assembling the paper pieces into tapered geometry as shown in FIG. 10. The tapered structure or geometry includes, but is not limited to, “point up” by 45 degree angled cut in porous material, “pointing” by different lengths of the layers of porous material, cylindrical shape with dimensions of radius and height, “arrow” by 45 degree angled cut in two side of porous material, “arrow” by different lengths of the layers of porous material in both side.

TABLE 2
Flow time of whole saliva solution as a function
of porous material cross-sectional area.

| Cross-sectional area (mm²) | Time to flow across device | |
|---|---|---|
| 3.7 | Does not fully flow across device | |
| 7.4 | ~5 | minutes |
| 14.8 | ~3.5 | minutes |

In one embodiment, the present invention provides a one-step system leveraging the porous material technology.

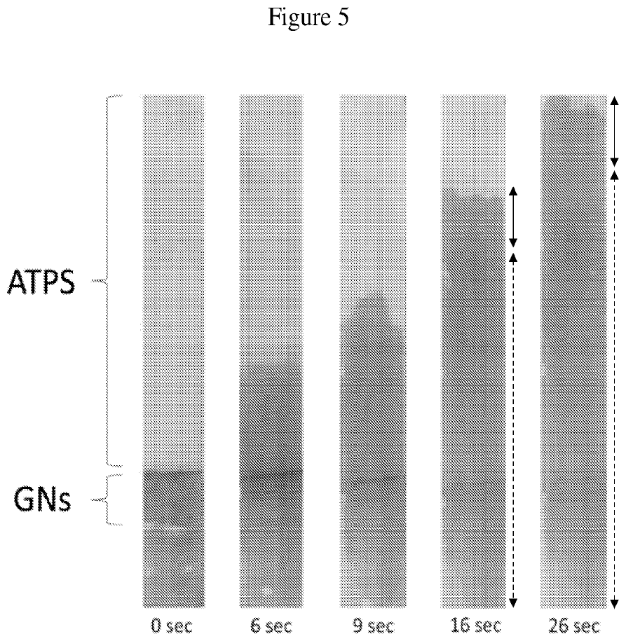

In one embodiment, with the optimized porous material and format described herein, the porous material module is further developed to improve the overall usability of the system. In one embodiment, in order to generate a true one-step test, all components of the ATPS are fully contained within the porous material module. In another embodiment, the end-users put the testing module into a fresh saliva specimen or plaque specimen and allow the test to develop for 15 minutes or less. In one embodiment, two elements of the device can be further optimized: (i) method of dehydrating the ATPS components and (ii) ATPS components and solubilizing conditions for achieving a phase separation after dehydration and rehydration. First, certain dehydration methods were found to increase or decrease the volumetric flow rate within the present porous material. Secondly, a dehydrated ATPS within the present porous material could actually induce phase separation. However, since solubilization of ATPS components is not instant, extensive screening was performed to identify the ideal conditions for this to occur and phase separation using dehydrated ATPS. FIG. 5 shows one screening result which shows the spontaneous phase separation of a solution using a paper strip with dehydrated ATPS components.

In one embodiment, the invention improves the detection limit for SM using a one-step porous material/LFA test.

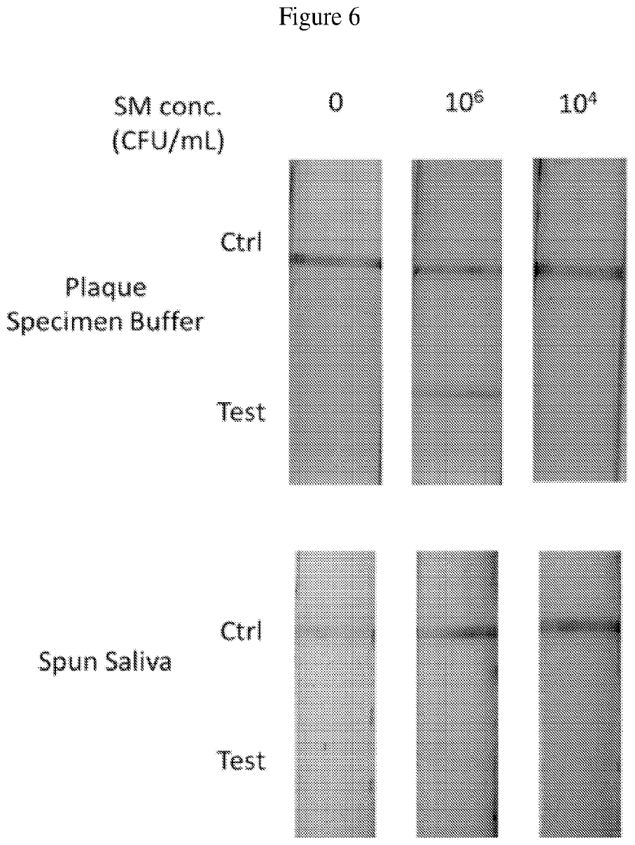

In one embodiment, the invention is able to achieve an improvement in the limit of detection using a one-step porous material/LFA test. In another embodiment, the invention can provide a 100-fold improvement up to a limit of detection of 104 CFU/mL, achieving the similar sensitivity of a two-step system but without the need for liquid handling steps (FIG. 6). The enhanced sensitivity was achieved in both PSB and spun saliva, showing the versatility of the one-step approach. The present invention represents a major breakthrough, as the present porous material module is a first-in-class biomolecule concentration technology that could be easily integrated into a large range of LFA tests for minimal extra cost.

In one embodiment, the present invention integrates semi-quantitation into the one-step SM test for risk assessment.

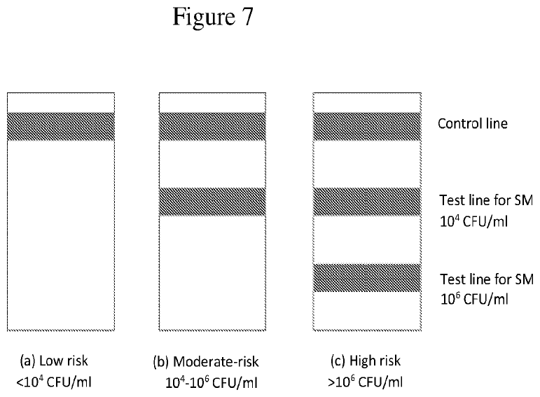

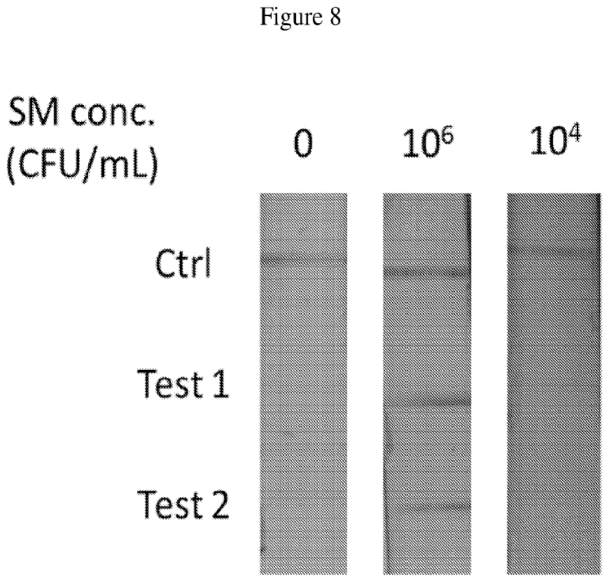

In one embodiment, the present invention represents a useful risk assessment tool in the overarching Caries Management by Risk Assessment (CAMBRA) guidelines. A semi-quantitative test that can distinguish certain levels of SM concentration can provide a more accurate risk assessment for clinicians. In one embodiment, a test strip is developed with a semi-quantitative feature built in that can distinguish between low, moderate, and high-risk SM infection levels (FIG. 7). These levels correspond to SM burdens of <10⁵ CFU/mL, <10⁶ but >10⁵ CFU/mL, and >10⁶ CFU/mL. The preliminary tests in spun saliva demonstrated the efficacy of this approach (FIG. 8). In another embodiment, the semi-quantitative test strip is developed to detect SM burdens <10³ CFU/mL, <10⁴ CFU/mL, >10⁷ CFU/mL, >10⁸ CFU/mL and other applicable concentrations.

In one embodiment, the present invention discloses a system for the detection and quantification of cariogenic bacteria in a saliva specimen from a subject, the system comprises:
(a) a purifying unit for removing interfering molecules proteins that interfere with detection of the cariogenic bacteria from the specimen,
(b) a concentrating module for concentrating the cariogenic bacteria,
(c) a conjugate pad comprising labeled biorecognition molecules, each of which comprises a label and a biorecognition molecule, where the cariogenic bacteria is bound by the biorecognition molecules, thereby leading to a complex of the cariogenic bacteria and the labeled biorecognition molecules, and,
(d) a detecting module for detecting the complex or the cariogenic bacteria, where the detecting module comprises a membrane covered by a control line and one or more test lines,
   and the concentrating module comprises a porous material precoated with components of an Aqueous Two-Phase System (ATPS), when a solution containing the cariogenic bacteria flows through the porous material, two separated phases are formed, and the cariogenic bacteria are concentrated in one of the two separated phases, thereby leading to a concentrated phase which continues to flow through the detecting module; and positive results on the control line and one or more test lines indicate the presence of the cariogenic bacteria in the subject or indicate the concentration of the cariogenic bacteria in the specimen.

In one embodiment, the purifying unit comprises a purifying agent that reacts with the interfering molecules proteins to form a precipitate, the precipitate is retained in the purifying unit and does not migrate with liquid flow or the precipitate does not interfere with the detection.

In one embodiment, the purifying agent includes but is not limited to trichloroacetic acid (TCA), TCA/acetone/dithiothreitol (DTT) mixture, TCA/acetone/2-mercaptoethanol mixture, acetone, and alcohol.

In one embodiment, the cariogenic bacteria include but are not limited to Streptococcus mutans (SM), Genus lactobacillus, Aggregatibacter actinomycetemcomitans, Porphyromonas gingivalis, Tannerella forsythia (formerly Bacteroides forsythus), Treponema denticola, Fusobacterium nucleatum, Prevotella intermedia, Prevotella nigrescens, and Eikenella corrodens.

In one embodiment, the biorecognition molecules include but are not limited to antibodies, aptamers, and molecular beacons.

In one embodiment, the porous materials include but are not limited to fiber-glass paper, cotton-based paper, single-layer matrix paper, and polyolefin foam pad.

In one embodiment, the ATPS components include but are not limited to polymers, salts, and surfactants.

In one embodiment, the polymers include but are not limited to polyalkylene glycols, poly(oxyalkylene)polymers, poly(oxyalkylene)copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, poly N-isopropylacrylamide, polyethylene glycol, polypropylene glycol, and dextran.

In one embodiment, the salts include but are not limited to kosmotropic salts, chaotropic salts, inorganic salts having a cation of trimethyl ammonium, triethyl ammonium, tripropyl ammonium, tributyl ammonium, tetramethyl ammonium, tetraethyl ammonium, tetrapropyl ammonium or tetrabutyl ammonium, and an anion of phosphate, sulphate, nitrate, chloride or hydrogen carbonate, NaCl, Na₃PO₄, K₃PO₄, Na₂SO₄, potassium citrate, (NH₄)₂SO₄, sodium citrate, sodium acetate, ammonium acetate, and any combinations thereof.

In one embodiment, the surfactants include but are not limited to nonionic surfactants, detergents, Triton-X, Igepal CA-630, and Nonidet P-40.

In one embodiment, the one or more test lines indicate a low, moderate or high risk of a dental disease or condition associated with said cariogenic bacteria in the subject.

In one embodiment, one or more test lines are pre-fixed with one or more antibodies or antigens at different concentrations, and the one or more antibodies or antigens specifically bind with the cariogenic bacteria or their complexes with the labeled biorecognition molecules.

In one embodiment, the system detects Streptococcus mutans at concentrations as low as 10⁴ CFU/ml.

In one embodiment, the present invention discloses a method of semi-quantitatively detecting a risk of a dental disease or condition associated with cariogenic bacteria in a subject, the method comprises the steps of:
(a) removing interfering molecules proteins from a saliva specimen from the subject in a purifying unit;
(b) passing a solution containing the cariogenic bacteria from step (a) through a concentrating module comprising a porous material precoated with components of an Aqueous Two-Phase System (ATPS), when the solution flows through the porous material, two separated phases are formed, and the cariogenic bacteria are concentrated in one of the two phases, thereby resulting in a concentrated solution,
(c) allowing the concentrated solution to migrate to a conjugate pad comprising labeled biorecognition molecules, each of which comprises a label and a biorecognition molecules, and the cariogenic bacteria is bound by said biorecognition molecules, thereby obtaining a complex of the labeled biorecognition molecules and the cariogenic bacteria, and
(d) detecting the complex or the cariogenic bacteria on a testing module comprising a membrane covered with a control line and one or more test lines, where positive results on said control line and one or more test lines indicate the presence of the cariogenic bacteria or a risk of a dental disease or condition associated with the cariogenic bacteria in the subject, or indicate the concentration of the cariogenic bacteria in the specimen.

In one embodiment, the purifying unit comprises a purifying agent that forms a precipitate by reacting with the interfering molecules and the precipitate does not migrate with liquid flow, or the precipitate does not interfere with the detection.

In one embodiment, the purifying agent includes but is not limited to TCA, TCA/acetone/dithiothreitol (DTT) mixture, TCA/acetone/2-mercaptoethanol mixture, acetone, and alcohol.

In one embodiment, the cariogenic bacteria include but are not limited to Streptococcus mutans (SM), Genus lactobacillus, Aggregatibacter actinomycetemcomitans, Porphyromonas gingivalis, Tannerella forsythia (formerly Bacteroides forsythus), Treponema denticola, Fusobacterium nucleatum, Prevotella intermedia, Prevotella nigrescens, and Eikenella corrodens.

In one embodiment, the ATPS components include but are not limited to polymers, salts, and surfactants.

In one embodiment, the polymers include but are not limited to polyalkylene glycols, poly(oxyalkylene)polymers, poly(oxyalkylene)copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, poly N-isopropylacrylamide, polyethylene glycol, polypropylene glycol, and dextran.

In one embodiment, the salts include but are not limited to kosmotropic salts, chaotropic salts, inorganic salts having a cation of trimethyl ammonium, triethyl ammonium, tripropyl ammonium, tributyl ammonium, tetramethyl ammonium, tetraethyl ammonium, tetrapropyl ammonium or tetrabutyl ammonium, and an anion of phosphate, sulphate, nitrate, chloride or hydrogen carbonate, NaCl, Na₃PO₄, K₃PO₄, Na₂SO₄, potassium citrate, (NH₄)₂SO₄, sodium citrate, sodium acetate, ammonium acetate, and any combinations thereof.

In one embodiment, the surfactants include but are not limited to nonionic surfactants, detergents. Triton-X, Igepal CA-630, and Nonidet P-40.

In one embodiment, the method detects Streptococcus mutans at concentrations as low as 10⁴ CFU/ml.

In one embodiment, one or more test lines are pre-fixed with one or more antibodies or antigens with different concentrations, and the one or more antibodies or antigens specifically bind with the cariogenic bacteria or their complex with the labeled biorecognition molecules.

This invention will be better understood by reference to the examples which follow. However, one skilled in the art will readily appreciate that the examples provided are merely for illustrative purposes and are not meant to limit the scope of the invention which is defined by the claims following thereafter.

### EXAMPLES

### Example 1—Remove Interfering Proteins from Saliva

0.5 ml of whole saliva specimens was mixed with 0.5 ml of TCA (20% w/v) and the mixture was vortexed to mix thoroughly and allowed to precipitate for 2 hour at −20° C. This was followed by centrifugation at 15,000 rpm, 4° C. for 20 min. The supernatant was collected. Sodium tetraborate was added to neutralization the supernatant until slightly alkaline such as a pH value of 7.5. The supernatant can then be concentrated by lyophilizer or ATPS system as demonstrated in FIGS. 1A-1B if needed. Any existence of protein in supernatant can be detected by UV at 280 nm. In Example 1, no reading can be observed at UV at 280 nm for the supernatant, it implies that all the protein were successfully removed from saliva.

### Example 2—Use ATPS to Concentrate the Saliva Supernatant

The supernatant prepared in Example 1 (1 ml) was added to 1 ml ATPS components comprising 25% PEG and 7.2% Potassium phosphat. After a thorough vortex, the mixture was allowed to separate. About 10 minutes later, the phase was separated as demonstrated in FIG. 1B. In addition, after the phase separation, the volume ratio of the top phase to the bottom phase changed from 1:1 to 9:1, and target molecules (shown in purple) partitioned in the bottom phase with a 5-fold concentration.

### Example 3—Detection of S. mutans by LFA in Patient Samples

Preparation of sample pad for LFA: Fiberglass porous paper sheets were cut into 0.5 cm×4 cm rectangles. The formulated ATPS components, 20% (w/w) PEG and 18.5% (w/w) potassium phosphate were pipetted onto the fiberglass porous paper. The above porous papers with ATPS were then dried in a lyophilizer for 2 hours first. Pieces were then stacked (four strips per stack) and were further cut into a tapered shape so that it forms a 45-degree angle at one end. In one embodiment, the tapered papers were assembled together so that the direction of flow is vertical to the horizontal line and the taper is ‘pointing’up’, as shown in the top-right image of FIG. 10. In one embodiment, the tapering is enabled by the 45 degree angled cuts in each layer of the porous material. In one embodiment, the cross-sectional area is about 14.8 mm².

In one embodiment, a PEG solution (in DI H₂O) was added to each porous material. 50 μl of a Tris-buffered solution containing 2% bovine serum albumin (BSA), and 0.1% PEG, 20 mM Tris pH 7.5 respectively) was added immediately after the addition of the first solution. The ATPS within porous papers were then placed in an indicator-containing (colloidal gold) buffer solution in PBS (overall pH 7.4), resulting in capillary action-mediated flow.

Preparation of LFA test strip: 1) Anti-Streptococcus mutans antibody (IgG) at a concentration of 1 mg/mL (supplied by abcam, ab31181), and 2) Protein (Bovine Serum Albumin, BSA) at a concentration of 0.2 mg/ml were added on the test strip. Colloidal gold nanoparticles were conjugated to the anti-Streptococcus mutans antibody as directed by manufacturer instructions. This conjugate was then dried onto the conjugating pad material using a lyophilizer. The absorbent pad consisted of untreated paper.

The LFA test strip was integrated with sample pad, i.e., the porous device/component with the dehydrated ATPS components and the phase separation behavior modifying agent. The porous device/component and the LFA component are placed into an appropriate housing such that the components are held in place.

Detection using LFA: The fiberglass porous paper dehydrated with ATPS components were dipped into saliva prepared in Example 2 which was in a PBS buffer solution at pH 7.4. Whole saliva without treatment was used for comparison. After the saliva solution flowed through the porous material and through the LFA test for 2 min, another 2 mins was needed to develop the test results. The detection limit was observation. The result is summarized in Table 3 below.

TABLE 3
Detection limit of Streptococcus mutans

| Sample | Detection limit |
|---|---|
| Whole saliva without treatment | 10⁶ |
| Saliva treated with TCA | 10⁴ |

1. A system for the detection and quantification of cariogenic bacteria in a saliva specimen from a subject, said system comprising:
(a) a purifying unit for removing interfering molecules that interfere with detection of said cariogenic bacteria from said specimen,
(b) a concentrating module for concentrating said cariogenic bacteria,
(c) a conjugate pad comprising labeled biorecognition molecules, each of which comprises a label and a biorecognition molecule, wherein said cariogenic bacteria is bound by said biorecognition molecules, thereby leading to a complex of said cariogenic bacteria and said labeled biorecognition molecules, and,
(d) a detecting module for detecting said complex or said cariogenic bacteria, wherein said detecting module comprises a membrane covered by a control line and one or more test lines,
wherein said concentrating module comprises a porous material precoated with components of an Aqueous Two-Phase System (ATPS), wherein when a solution containing said cariogenic bacteria flows through said porous material, two separated phases are formed, wherein said cariogenic bacteria are concentrated in one of the two separated phases, thereby leading to a concentrated phase which continues to flow through said detecting module; and
wherein positive results on said control line and one or more test lines indicate the presence of said cariogenic bacteria in said subject or indicate the concentration of said cariogenic bacteria in said specimen.

2. The system of claim 1, wherein said purifying unit comprises a purifying agent that reacts with said interfering molecules to form a precipitate, said precipitate is retained in the purifying unit and does not migrate with liquid flow or said precipitate does not interfere with the detection.

3. The system of claim 2, wherein said purifying agent is selected from the group consisting of trichloroacetic acid (TCA), TCA/acetone/dithiothreitol (DTT) mixture, TCA/acetone/2-mercaptoethanol mixture, acetone, and alcohol.

4. The system of claim 1, wherein said cariogenic bacteria are selected from the group consisting of Streptococcus mutans (SM), Genus lactobacillus, Aggregatibacter actinomycetemcomitans, Porphyromonas gingivalis, Tannerella forsythia (formerly Bacteroides forsythus), Treponema denticola, Fusobacterium nucleatum, Prevotella intermedia, Prevotella nigrescens, and Eikenella corrodens.

5. The system of claim 1, wherein said biorecognition molecules are selected from the group consisting of antibodies, aptamers, and molecular beacons.

6. The system of claim 1, wherein the porous material is selected from the group consisting of fiber-glass paper, cotton-based paper, single-layer matrix paper, and polyolefin foam pad.

7. The system of claim 1, wherein said ATPS components are selected from the group consisting of polymers, salts, and surfactants.

8. The system of claim 7, wherein said polymers are selected from the group consisting of polyalkylene glycols, poly(oxyalkylene)polymers, poly(oxyalkylene)copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, poly N-isopropylacrylamide, polyethylene glycol, polypropylene glycol, and dextran.

9. The system of claim 7, wherein said salts are selected from the group consisting of kosmotropic salts, chaotropic salts, inorganic salts having a cation of trimethyl ammonium, triethyl ammonium, tripropyl ammonium, tributyl ammonium, tetramethyl ammonium, tetraethyl ammonium, tetrapropyl ammonium or tetrabutyl ammonium, and an anion of phosphate, sulphate, nitrate, chloride or hydrogen carbonate, NaCl, Na₃PO₄, K₃PO₄, Na₂SO₄, potassium citrate, (NH₄)₂SO₄, sodium citrate, sodium acetate, ammonium acetate, and any combinations thereof.

10. The system of claim 7, wherein said surfactants are selected from the group consisting of nonionic surfactants, detergents, Triton-X, Igepal CA-630, and Nonidet P-40.

11. The system of claim 1, wherein said one or more test lines indicate a low, moderate or high risk of a dental disease or condition associated with said cariogenic bacteria in said subject, wherein said one or more test lines are pre-fixed with one or more antibodies or antigens at different concentrations, wherein said one or more antibodies or antigens specifically bind with said cariogenic bacteria or their complexes with said labeled biorecognition molecules, wherein said system detects Streptococcus mutans at concentrations as low as 10⁴ CFU/ml.

12. (canceled)

13. (canceled)

14. A method of semi-quantitatively detecting a risk of a dental disease or condition associated with cariogenic bacteria in a subject, said method comprising the steps of:
(a) removing interfering molecules from a saliva specimen from said subject in a purifying unit,
(b) passing a solution containing said cariogenic bacteria from step (a) through a concentrating module comprising a porous material precoated with components of an Aqueous Two-Phase System (ATPS), wherein when said solution flows through said porous material, two separated phases are formed, and the cariogenic bacteria are concentrated in one of the two phases, thereby resulting in a concentrated solution,
(c) allowing said concentrated solution to migrate to a conjugate pad comprising labeled biorecognition molecules, each of which comprises a label and a biorecognition molecules, wherein said cariogenic bacteria is bound by said biorecognition molecules, thereby obtaining a complex of said labeled biorecognition molecules and said cariogenic bacteria, and
(d) detecting said complex or said cariogenic bacteria on a testing module comprising a membrane covered with a control line and one or more test lines, wherein positive results on said control line and one or more test lines indicate the presence of said cariogenic bacteria or a risk of a dental disease or condition associated with the cariogenic bacteria in said subject or the concentration of said cariogenic bacteria in said specimen.

15. The method of claim 14, wherein said purifying unit comprises a purifying agent that forms a precipitate by reacting with said interfering molecules and said precipitate does not migrate with liquid flow, or said precipitate does not interfere with the detection.

16. The method of claim 15, wherein said purifying agent is selected from the group consisting of TCA, TCA/acetone/dithiothreitol (DTT) mixture, TCA/acetone/2-mercaptoethanol mixture, acetone, and alcohol.

17. The method of claim 14, wherein said cariogenic bacteria are selected from the group consisting of Streptococcus mutans (SM), Genus lactobacillus, Aggregatibacter actinomycetemcomitans, Porphyromonas gingivalis, Tannerella forsythia (formerly Bacteroides forsythus), Treponema denticola, Fusobacterium nucleatum, Prevotella intermedia, Prevotella nigrescens, and Eikenella corrodens.

18. The method of claim 14, wherein said ATPS components are selected from the group consisting of polymers, salts, and surfactants.

19. The method of claim 18, wherein said polymers are selected from the group consisting of polyalkylene glycols, poly(oxyalkylene)polymers, poly(oxyalkylene)copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, poly N-isopropylacrylamide, polyethylene glycol, polypropylene glycol, and dextran.

20. The method of claim 18, wherein said salts are selected from the group consisting of kosmotropic salts, chaotropic salts, inorganic salts having a cation of trimethyl ammonium, triethyl ammonium, tripropyl ammonium, tributyl ammonium, tetramethyl ammonium, tetraethyl ammonium, tetrapropyl ammonium or tetrabutyl ammonium, and an anion of phosphate, sulphate, nitrate, chloride or hydrogen carbonate, NaCl, Na₃PO₄, K₃PO₄, Na₂SO₄, potassium citrate, (NH₄)₂SO₄, sodium citrate, sodium acetate, ammonium acetate, and any combinations thereof.

21. The method of claim 18, wherein said surfactants are selected from the group consisting of nonionic surfactants, detergents, Triton-X, Igepal CA-630, and Nonidet P-40.

22. The method of claim 14, wherein said method detects Streptococcus mutans at concentrations as low as 10⁴ CFU/ml, wherein said one or more test lines are pre-fixed with one or more antibodies or antigens with different concentrations, wherein said one or more antibodies or antigens specifically bind with the cariogenic bacteria or their complex with said labeled biorecognition molecules.

23. (canceled)
